(19) United States
(12) Patent Application Publication
(10) Pub. No.: US 2008/0255104 A1
(43) Pub. Date: Oct. 16, 2008

(54) Nsaid Compositions

(75) Inventors: Fahkreddin Jamali, Edmonton (CA); Aghazadeh Habashi, Edmonton (CA)

Correspondence Address: WILLIAM J BUNDREN;THE LAW OFFICE OF WILLIAM J BUNDREN, 734 LaRue Road, Millersville, MD, 21108, US

(73) Assignee: EQUITECH CORPORATION, Edmonton, AB (CA)

(21) Appl. No.: 11/568,551

(22) PCT Filed: May 4, 2005
(86) PCT No.: PCT/CA2005/000684

§ 371 Date: Jun. 30, 2008

Related U.S. Application Data
(60) Provisional application No. 60/567,436, filed on May 4, 2004.

(51) Int. Cl. A61K 31/5415; A61K 31/192; A61P 29/00

(52) U.S. Cl. 514/226.5; 514/570

(57) ABSTRACT

The invention is directed to a composition and method for treating acute pain using a composition comprising one or more NSAID's, a metasilicate and optionally a fatty acid ester resulting in increased absorption of poorly soluble active NSAID's and increased absorption in suppressed vagal systems. The preferred composition comprises meloxicam on a metasilicate matrix; and one or more of the following: sodium bicarbonate, Gelucire®, and tartaric acid.

### BACKGROUND OF THE INVENTION

1. Field of the Invention

The present invention is directed to pharmaceutical compositions including a metasilicate and a fatty acid ester, increased absorption of poorly soluble active agents, and increased absorption in suppressed vagal systems. One of the poorly soluble NSAID active agents, meloxicam, is a potent and well-tolerated anti-inflammatory, analgesic, and anti-pyretic compound.

2. Description of Related Art

Under certain pathophysiological conditions such as stress, trauma, and pain, absorption of drugs through the stomach and intestine may be impaired. This is believed to be due to suppression of the vagal nervous system, two of the consequences of which include delayed gastric emptying and reduced secretion of gastrointestinal fluid. For example, in the treatment of acute pain, rapid absorption of orally administered analgesics is desirable. For non-steroidal anti-inflammatory drugs (NSAIDs), such as meloxicam, there appears to be a positive relationship between plasma drug concentration and analgesic activity. Any delay in absorption or reduction in the circulating drug concentration may result in treatment failure or in reduced activity of the analgesic. One skilled in the art readily recognizes that analgesic formulations with enhanced absorption rates are expected to be more effective in treating acute pain. Absorption rates may be enhanced by improving one or more of a number of factors, including but not limited to increasing the rate or speed of disintegration, increasing the rate or speed of dissolution, changing the pH of the stomach, increasing the amount of water in the stomach, and altering the solubility of the active agent.

However, none of the widely available solid dosage forms of NSAIDs have been claimed to be superior over the products of the same drug with respect to onset of action. This is despite differences in apparent rate of absorption usually measured in healthy volunteers. It appears that rapid absorption observed in healthy subjects does not necessarily result in a quick onset of action in patients experiencing pain.

Jamali & Kunz, Brit J. Clin. Pharmacol., 47:391-396 (1999) have reported that, using dental surgery as a marker of pain, pain or its associated trauma causes reduced rate of absorption of ibuprofen. The publication details the absorption rates for two doses of ibuprofen, 200 mg and 600 mg. Surgery resulted in a two hour delay in the mean time to peak concentration, significant decreases in serum ibuprofen concentrations following both doses, and a fall to sub-optimal serum concentrations following the 200 mg dose.

For example, during the first two hours after the 200 mg dose, dental extraction resulted in a significant reduction of the area under serum drug concentration (AUC 0-2h, mg/L−1/h) from 5.6∀ 2.9 to 1.6∀1.8 (p<0.01) and from 5.5∀ 3.0 to 2.1∀ 2.0 (p<0.05) for S and R-ibuprofen, respectively. Similar observations were made following the 600 mg dose for AUC0-2h of S-ibuprofen (from 14.2 ∀ 6.1 to 7.2 ∀ 5.5 mg. L−1·h, p<0.05) with no significant difference for R-ibuprofen (form 14.4 ∀ 9.5 to 5.8 ∀ 7.1). AUC0-6h was also significantly reduced by surgery.

The publication concludes that wisdom tooth removal, as an example of a person in pain, resulted in substantial decreases in the serum concentration of ibuprofen enantiomers and an increase in the period to peak concentration. Thus, dental patients may experience a delayed response and possible treatment failure when taking ibuprofen for pain relief after surgery.

The observed reduced absorption is believed to be caused by suppression of the vagal nervous system, causing reduced gastric juice secretion and motility, both of which are associated with decreased absorption of NSAIDs. Sufficient fluid and a rather quick exit from stomach (hence entry to small intestine, the major site of absorption) is needed for efficient absorption.

The problem of decreased absorption in vagally suppressed mammals is further exacerbated by the relative insolubility of NSAIDs in an aqueous or gastric (acidic) environment. Finally, there is growing evidence that these conditions—namely, reduction in stomach motility, stomach secretion diminution, and reduced absorption—appear to be present in the elderly, or what shall be termed herein, the geriatric stomach.

There are many different approaches to increasing the bioavailability of an active agent, including but not limited to regulating a tablet's disintegration and, post-disintegration, the active agent's dissolution. To enhance absorption, after disintegration, the active ingredient must become available (e.g., freed from the structure of the tablet), be reasonably dissolved in the gut fluid, and stay soluble until absorbed. To speed up disintegration and dissolution, some prior art formulations, e.g., PCT/EP97/00841, incorporate an alkali metal bicarbonate into the formulation containing acidic active agents (such as ibuprofen and meloxicam). Upon exposure to an aqueous medium, the bicarbonate and acid interacts and produces carbon dioxide (CO₂). This reaction results in rapid disintegration of the solid dosage form. In addition, the reaction results in conversion of the poorly soluble acid (active agent) to its soluble salt. Alkali metal carbonates and bicarbonates are soluble materials which have previously been proposed for use in effervescent tablets, for example to react with the acid component in an effervescent couple (see for example WO 94/10994) or to prevent initiation of the effervescent reaction e.g. during storage. Effervescent tablets disintegrate by means of the reaction between acid and base, particularly in the presence of water, leading to the production of carbon dioxide. In these formulations, disintegration and dissolution occur prior to administration or ingestion of the tablet, e.g., in a cup of water. As such a solution of the active agent rather than a solid dosage form is administered to the patient.

Furthermore, in the gut, specifically in a vagally suppressed system, the active agent in its salt form may subsequently precipitate out of solution into its less soluble acid form. This results in reduced absorption. To address this issue, it is known to incorporate one or more anti-precipitation agents in the pharmaceutical formulation, but these agents do not always result in enhanced absorption directly or to the expected amount of increased absorption.

Some prior art formulations, e.g., PCT/EP97/00841, incorporate an alkali metal bicarbonate into the formulation to enhance the compressibility of the solid dosage form. These formulations include ibuprofen as the active agent, the bicarbonate as a compressibility enhancer, a compressible filler, and a disintegrant (preferably croscarmellose sodium or sodium starch glycollate).

In accordance with the present invention, the tablet is specifically designed to be swallowed intact, e.g., prior to disintegration, and any effervescent reaction that might exist, occurs in the stomach. This contrasts with effervescent systems in which the active agent is solubilized or put into solution using an effervescent reaction, and then the solubilized active agent is ingested.

Gupta et al (2001) teaches that a solid dispersion containing Gelucire® and Neusilin® enhances the dissolution rate of BAY 12-9566, a naproxen-containing composition [Gupta et al., Pharm. Dev. Technol. 6(4):563-72 (2001)]. Kinoshita et al (2002) teaches that the dissolution rate of a poorly water-soluble drug, 3-bis(4-methoxyphenyl) methylene-2-indolinone (TAS-301), is improved when it is melt-adsorbed on a porous calcium silicate. [Kinoshita et al., J. Pharm. Sci. 91(2):362-70 (2002)]. Gupta et al (2003) teaches that the dissolution rate of carboxylic acid-containing drugs from Neusilin®-Gelucire® formulations is enhanced due to their hydrogen binding to Neusilin®; it is believed that Neusilin® renders the drug amorphous [Gupta, et al., J. Pharm. Sci. 92(3):536-51 (2003)].

### SUMMARY OF THE INVENTION

It is desirable to provide an active agent formulation that can deliver the active agent into the blood stream under normal (e.g., non-pain) and suppressed nervous vagal system (e.g., in pain) conditions. The preferred active agents are NSAIDs, specifically, meloxicam.

It would be advantageous to provide a composition having enhanced absorption of NSAIDs, which tend to be poorly water soluble, as well as providing an improved concentration of the drug at the cellular level at the site of its action. Further, it is highly desirable to provide a formulation that increases the bioavailability of an active agent faster. It would also be advantageous to provide a method and composition for increasing the absorption rate of such poorly water-soluble active agents by increasing the disintegration efficiency of the composition in tablet form, by accelerating the time and speed of the tablet disintegrating into molecules in solution, and by increasing the speed by which active agent is available in solution for absorption.

NSAIDs (or aspirin-like drugs) are typically categorized into six structural groups. One class, the oxicams, are acid enolcarboxamides, include but are not limited to piroxicam, tenoxicam, lomoxicam, and meloxicam, and the pharmaceutically acceptable salts thereof. The terms “NSAIDs” or “NSAID substances” are used herein to designate a group of drugs that belongs to non-steroid anti-inflammatory drug substances and pharmaceutically acceptable salts, prodrugs and/or complexes thereof as well as mixtures thereof.

Meloxicam is an antirheumatic agent belonging to a class of cyclooxygenase inhibitors (COX). Meloxicam has been shown to have a selective inhibitory effect on the isoenzyme COX-2 and consequently a reduced risk of undesirable gastrointestinal side effects. Meloxicam is an NSAID with the structural type of an enolic acid and exhibits a distinctly pH-dependent solubility. The minimum solubility in buffered aqueous systems is found at pH values from 2-4. The solubility in this pH range is less than 0.5 .mu.g/ml (Luger P., Daneck K., Engel W., Trummlitz G., Wagner K., Structure and physicochemical properties of meloxicam, a new NSAID, Eur. J. Pharm. Sci. 4 (1996), 175-187). Suitable dispersion media for a liquid oral suspension of meloxicam according to prior art formulations are therefore physiologically acceptable aqueous buffer systems with a pH in the range from 2-4, mixtures thereof or mixtures thereof with other physiologically acceptable liquids which are additionally suitable for improving specific properties of the meloxicam suspension.

Diseases suitable for treatment using an NSAID include but are not limited to pain, fever and inflammation of a variety of conditions including rheumatic fever, symptoms associated with influenza or other viral infections, common cold, low back and neck pain, dysmenorrhea, headache, toothache, sprains and strains, myositis, neuralgia, synovitis, arthritis, including rheumatoid arthritis degenerative joint diseases (osteoarthritis), gout and ankylosing spondylitis, bursitis, burns, injuries.

A critical factor relating to the use of meloxicam to treat the above disorders concerns, as noted above, improving the onset of action of meloxicam, particularly in the treatment of pain. This issue partially concerns improving the amount and speed of achieving a certain blood serum level of meloxicam. It is believed that rapid disintegration of a formulation, primarily in the stomach, releases the drug into the body more quickly, thereby leading to a more rapid onset of therapeutic action, as compared with a standard dosage form or with dosage forms calibrated against healthy individuals. Accordingly, it is desired to produce a solid dosage form for oral administration adapted to disintegrate quickly in the gastro-intestinal tract. It is also preferred that the dosage form is manufactured by compression on standard tabletting machines.

In accordance with one embodiment of the present invention, the composition contains an NSAID, preferably meloxicam; and an alkalating agent, such as a metasilicate. The composition may also include a disintegration and dissolution agent, such as a bicarbonate, preferably sodium bicarbonate; an ester of a fatty acid as an anti-precipitation agent; and tartaric acid as an additional excipient. The composition may optionally also include starch. These ingredients are formed into a tablet or solid form, a tablet having enhanced disintegration into particles and subsequently enhanced dissolution of the particles into dispersed molecules in solution.

In accordance with the present invention, the bicarbonate is a disintegrator or disintegrating agent that increases the solubility of the NSAID. The anti-precipitant provides an interface between lipid and aqueous phases (i.e., under gastric conditions) and prevents and/or reduces precipitation of the meloxicam in the gastric environment. While not intending to be limited to a particular mechanism of action, the inventor believes that the bicarbonate increases solubility by promoting the formation of sodium salts that are readily converted to an active form; most NSAIDS precipitate under gastric conditions, so the anti-precipitation agent prevents precipitation by increasing the solubility of the NSAID in the gastric environment. The inclusion of anti-precipitants, such as Gelucire® and other similar compounds, may be desirable in a composition of the present invention in order to prevent or reduce the amount of active ingredient that precipitates in an acidic environment.

The compositions and methods of the present invention achieve chemically what happens biologically when NSAIDS are administered and absorbed in healthy subjects. Biologically, the stomach has a certain amount of movement or motility, as well as gastric juice that contribute to a tablet disintegrating into particles, and then dissolving into molecules.

In a vagally suppressed human, i.e., a human in pain and/or the geriatric stomach, the motility, amount of available water, and gastric juice extraction (or secretion) are reduced. This results in delayed absorption. The present invention accelerates the time line of disintegration into particle form by chemically mimicking the agitation provided by the motility function, by initiating the disintegration from tablet form into particles as soon as the tablet is exposed to a very limited amount of fluid. In the presence of some moisture, the incorporated bicarbonate starts reacting with the meloxicam. This results in the larger solid particles breaking down, enhancing solubility, and providing a greater amount of active agent earlier in the process, thereby accelerating the absorption rate, and thereby providing more relief, faster.

The compositions and methods of the present invention achieve this result by surrounding, capturing, or formulating active agent particles, such as meloxicam, in a matrix or the like of a metasilicate, such as Neusilin®. Neusilin® is a porous magnesium aluminosilicate capable of enhancing dissolution rate of poorly soluble drugs. Other porous silicates are expected to do the same.

The composition may further include a disintegrating agent that, that, upon exposure to an aqueous environment, promotes the break-up of the tablet into smaller particles of active agent, thereby increasing the availability of the active agent for absorption.

As noted above, it is highly desirable to increase the solubility of the poorly soluble active ingredient, preferably after disintegration of the tablet. In accordance with the present invention, it has been found that both a metasilicate and a fatty acid ester can individually increase the solubility of certain drugs. Surprisingly, however, the combination of these two ingredients increases the solubility of meloxicam to an extent that exceeds expectations.

The solid dosage forms according to the invention are adapted for direct administration to a patient to obtain the desired therapeutic effect. They are not intended to be dissolved or dispersed in water prior to administration. Furthermore, the compressed dosage forms according to the present invention need no further processing after compression of a composition comprising a mixture of the ingredients to produce a solid dosage form.

As noted above, both Gelucire® and Neusilin® are known by those skilled in the art to increase the solubility of a poorly soluble drug. What was not known, and what is a novel feature of the present invention, is that the solubility of meloxicam is increased far beyond what would have been expected for Gelucire® and Neusilin® alone.

In the formulations of the present invention, all show improved bioavailability of meloxicam. It is believed that the increased rate of absorption of meloxicam can be attributed to the bicarbonate and tartaric acid. It is also believed that the increased extent of absorption of meloxicam can be attributed to the Neusilin® and the Gelucire®.

The accompanying drawings show illustrative embodiments of the invention from which these and other of the objectives, novel features and advantages will be readily apparent.

### DESCRIPTION OF THE DRAWINGS

FIG. 1 shows the plasma concentration time curve after the oral administration of a composition of the present invention (Formulation 1) versus Mobicox®.

FIG. 2 shows that the endothermic peak for meloxicam is altered when formed into a formulation of the present invention.

FIG. 3 shows the plasma concentration profile for the oral administration of Mobicox® in healthy and pain model rats.

FIG. 4 shows the comparative dissolution profiles for two tablet formulations of meloxicam: a composition of the present invention (Zag 32″) and a commercially available formulation (Mobicox®).

FIG. 5 compares the oral availability for a composition of the present invention (Zag 32″) and a commercially available formulation (Mobicox®).

FIG. 6 shows the area under the curve (AUC) at 1 and 6 hours comparing a composition of the present invention (Zag 32″) and a commercially available formulation (Mobicox®).

FIG. 7 shows the solubility of meloxicam is simulated gastric fluid. PM=physical mixture; Gel=Gelucire®.

### DETAILED DESCRIPTION OF THE INVENTION

The present invention is a composition containing an NSAID as an active agent, said composition having increased absorption generally, and specifically in vagally suppressed systems. The preferred COX-2 inhibitor is meloxicam. The composition may comprise an NSAID such as meloxicam in a matrix or carrier, such as a porous silicate. The composition may further include a disintegration and dissolution agent, such as a bicarbonate; and an anti-precipitation agent. The composition may further include tartaric acid as an excipient. Examples of a porous carrier include, for instance, a porous metasilicate, including but not limited to aluminum magnesium metasilicate (available from Fuji Chemical Industry Co., Ltd. under the tradename Neusilin®).

The present invention is also a composition comprising meloxicam on a matrix comprising a metasilicate; and a disintegration and dissolution agent, such as a bicarbonate. The invention also includes a method of treating inflammation or alleviating pain comprising administering a composition as described.

The present invention is also a composition comprising meloxicam on a matrix comprising a metasilicate, a disintegration and dissolution agent, such as a bicarbonate, and an anti-precipitation agent. The preferred anti-precipitation agent is Gelucire. Such a composition is characterized by having increased absorption of the active agent, as compared to other compositions when the comparison assesses the absorption of the active agent under pain conditions.

In an exemplary preferred embodiment of the invention, the composition includes meloxicam in or on a metasilicate carrier; a fatty acid ester to aide in solubilization among other functions; a bicarbonate as a disintegration and/or dissolution agent; tartaric acid as an excipient; and maize starch as a disintegration agent.

The invention also includes a method of treating inflammation or alleviating pain comprising administering a composition as described.

The present invention is also any of the above compositions, further comprising one or more lubricating agents, one or ore binders, one or more additional disintegrating agents, one or more flow aids, and/or one or more colorants and/or flavorants.

The present invention is also a method for increasing the absorption of an NSAID-containing composition, said method comprising providing a composition, such as one of the compositions described above, whose ingredients are specifically formulated to increase absorption under pain conditions, i.e., in a vagally suppressed system. In preferred embodiments of the invention, the method includes increasing the absorption of meloxicam, typically both its rate and extent.

The present invention is also a method of treating chronic or acute pain in humans comprising administering a composition according to the present invention.

It will be appreciated that the present invention provides a method of treating inflammation, pain and pyrexia by administering a pharmaceutical composition comprising meloxicam, together with a pharmaceutically acceptable carrier to a mammal, e.g. a human, in need thereof.

The compositions and methods of the present invention are particularly suited to forming non-aqueous granulations and to solid non-effervescent dosage forms.

The present invention further relates to the use of the above composition to provide tablets and granules that are fast dissolving and fast acting. The granulation and tableting composition also includes normal excipients useful for the preparation of tablets.

The present invention is also a composition comprising an NSAID as an active agent, and a bicarbonate as a disintegrating agent. The composition may further comprise one or more of the following: one or more diluents or fillers; one or more binders or adhesives; one or more additional disintegrating agents; one or more lubricating agents; and one or more miscellaneous adjuncts, such as colorants and/or flavorants, any of said adjuncts being well known to those skilled in the art.

The compositions of the invention may contain about 1-99% by weight of an NSAID, such as meloxicam, preferably up to about 60% by weight, more preferably from about 15% to about 50% by weight; 1-99% by weight metasilicate, such as Neusilin®, preferably up to about 60% by weight, more preferably from about 15% to about 50% by weight; and 10-60% by weight of a bicarbonate, preferably between about 20% and 50%, and more preferably, between about 30% and 40%. In compositions that include an anti-precipitant, the anti-precipitant is present in an amount preferably up to about 50% by weight, more preferably from about 1% to about 30% by weight, and most preferably, from about 5% to about 7% by weight. The composition may also include up to about 30% by weight tartaric acid, preferably up to about 15%, more preferably between about 1% and about 10% by weight.

The compositions of the invention are generally prepared in unit dosage form. Preferably the unit dosage of meloxicam is in the range of 10-1200 mg in a pre-calculated amount to provide doses which are equivalent by weight to doses of for example 25 mg, 50 mg, 100 mg, 200 mg, 400 mg or 800 mg of meloxicam. The amount of e.g. an NSAID substance in a quick release composition according to the invention may be selected so that is corresponds to about 1 mg, 2 mg, 3 mg, 4 mg, 5 mg, 8 mg, 10 mg, 12 mg, 16 mg, 20 mg, 24 mg, 25 mg, 30 mg, 32 mg, 50 mg, 60 mg, 100 mg, 200 mg, 300 mg, 400 mg, 500 mg, 600 mg, 700 mg, 800 mg, 900 mg, 1 g, 1.1 g, 1.2 g, 1.3 g or 1.6 g of NSAID substance which are dosages generally known in the art.

A composition according to the invention may be produced in different series of dosage forms of e.g. 4 mg, 8 mg, 12 mg, 16 mg, 24 mg, 32 mg etc., each of the series having individual properties resulting from the design of modified release of the composition. Any desired total dosage can then be selected from the relevant dosage forms within each of the series.

The preferred dosage form according to the invention is in the form of a capsule, tablet, sachet etc. The size of the dosage form is adapted to the amount of the active drug substance contained in the composition.

The above suggested dosage amounts should not be regarded as a limitation of the scope of the invention as it is obvious for the skilled person that any desired amount of the active drug substance may be applied and is only limited by the size of the composition and the type of the active drug substance.

Any number of pharmaceutically active agents may be employed in the formulations of the present invention. These active agents may exist as either solids or liquids at standard temperature and pressure. Exemplary pharmaceutically active agents suitable for use herein include, but are not limited to, the non-steroidal anti-inflammatory agents such as piroxicam, indomethacin, fenoprofen, meloxicam, and ibuprofen. The preferred active agents are COX-2 inhibitors. In a preferred embodiment of the invention, the composition and method includes meloxicam as the active agent.

In the composition of the present invention, the active agent, preferably meloxicam, is carried on or in a carrier, such as the porous silicate noted above. Any porous silicate may be used in the practice of the present invention. The preferred silicates are metal silicates. The most preferred silicates are magnesium aluminosilicates, commercially available from the Fuji Chemical Industry Co. under the trademark Neusilin®.

In accordance with the present invention, the active agent is included in a composition that also includes both a porous silicate and a fatty acid ester. The fatty acid ester acts to increase the dissolution rate and to increase the solubility of the meloxicam. In accordance with the present invention, the fatty acid ester also acts as a mobility agent facilitating the interaction of the meloxicam with the silicate. The fatty acid ester may be any fatty acid ester that functions to increase the dissolution rate of an active agent, increase the solubility of a poorly soluble active agent, and/or acts as a mobility agent between the carrier and the active agent. Certain esters of natural vegetable oil fatty acids, for example, Gelucire®, are a fatty acid ester excipient that comprises one or more polyol esters and triglycerides of natural vegetable oil fatty acids. Gelucires® are glycerides and partial glycerides, and are commercially available under the trademark Gelucire® from Gattefosse Corporation, Hawthorne, N.Y. These excipients are available with varying physical characteristics such as melting point, HLB and solubilities in various solvents. The preferred Gelucire® is Gelucire® 44/14.

The bicarbonate can be any bicarbonate salt that is pharmaceutically acceptable, preferably sodium or potassium bicarbonate. The alkali metal carbonate or bicarbonate used in accordance with the present invention may suitably comprise sodium carbonate or bicarbonate or potassium carbonate or bicarbonate either alone or mixed together. Preferably, the alkali metal comprises sodium, thus sodium bicarbonate and sodium bicarbonate are preferred ingredients. The alkali metal carbonates may be supplied anhydrous or in varying degrees of hydration for example the monohydrate and decahydrate. Any of these forms may be used.

In therapeutic use, meloxicam may be administered orally, rectally, or topically, preferably orally or topically. Suitably the therapeutic compositions of the present invention may take the form of any of the known pharmaceutical compositions for oral, rectal, or topical administration. Pharmaceutically acceptable carriers suitable for use in such compositions are well known in the art of pharmacy.

Solid compositions for oral administration are preferred compositions of the invention and there are known pharmaceutical forms for such administration, for example tablets and capsules.

Within the context of the present description the identity of the components and amounts thereof refer to the weight and identity of the starting materials used in preparing the composition. It is possible that during preparation of the composition and/or tablets, some interaction or reaction may occur between two or more components. To the extent that such interaction or reaction occurs the present invention is intended to cover such occurrences.

Normal excipients useful in the preparation of the tablets include, but are not limited to: lubricants such as magnesium stearate, sodium stearyl fumarate and sodium benzoate; anti-adherents such as talc and polyethylenglycol; glidants such as colloidal silica; diluents such as dicalcium phosphate, cellulose (for example microcrystalline cellulose) and its derivatives, carbohydrates and polyalcohols such as saccharose, xylitol and lactose; disintegrants such as crosslinked vinylic polymers (such as crosslinked PVP), derivatives of starch and of cellulose such as sodium carboxymethyl-starch and sodium croscarmelose; wetting agents such as TWEEN 80® (Trademark registered by ICI of Americas for polysorbate) and sodium lauryl sulphate.

Suitable excipients and their amounts can be readily determined by those of ordinary skill in the art according to the methods normally used in pharmaceutical technology. However, in the present invention, it is important to avoid excipients that would cause a significant decrease in tablet dissolution rate. Further, excipients must allow a good workability during the manufacture of the tablet.

In preparing the tablet of the present invention it is preferable to prepare a meloxicam granulate, to mix it with the bicarbonate and the excipients, and then to compress. An exemplary method of preparing a composition of the present invention comprises dissolving meloxicam in an alkaline solution; mix with Neusilin®; change the pH (e.g., by adding glacier acid) so that the meloxicam re-crystallizes; and remove the solution. The meloxicam is thereby loaded on the Neusilin® matrix. It should then be dried, e.g., overnight.

The meloxicam loaded on the Neusilin® can then be mixed with one or more ingredients according to the invention. For example, it can be mixed with tartaric acid, corn starch, Gelucire®, sodium bicarbonate, microcrystalline cellulose, and sodium croscarmellose. The composition is then suitable for compressing into a tablet.

An exemplary solid composition according to the invention may include: 1-99% meloxicam (preferably 15-60%); 1-90% of a diluent (preferably 40-85%); 0.5-25% of a solubilizer (preferably 1-10%); 0.1-10% of a lubricating agent (preferably 0.5 to 5%); 1-50% of a disintegrating agent (preferably 2-20%); up to about 30% tartaric acid (preferably up to about 15%), and optionally, 0.1-15% of a binder. Optionally 0.1-10% of a flow aid may be added. It will be appreciated by those skilled in the art that a particular excipient may perform more than one function. For example maize starch may act as a diluent, a binder or as a disintegrating agent.

A preferred process for preparing a solid composition in tablet form comprises combining 10-90% of meloxicam with 1-90% of a diluent, optionally adding other pharmaceutically acceptable excipients selected from lubricating agents, disintegrating agents, binders, flow aids, oils, fats and waxes, mixing the ingredients with one another to form a uniform mixture, and compressing the mixture thus obtained to form tablets which may be optionally coated with a film coat or a sugar-coat. In a most preferred process for preparing a solid composition in tablet form, an active ingredient such as meloxicam is mixed with a bicarbonate, such as sodium bicarbonate under non-aqueous conditions. For example, in a conventional granulation step, meloxicam and sodium bicarbonate are combined using isopropyl alcohol as the diluent.

Preferably the diluent includes lactose, calcium phosphate, dextrin, microcrystalline cellulose, sucrose, starch, calcium sulphate, sodium bicarbonate, or mixtures thereof.

Preferably the lubricating agent includes magnesium stearate, stearic acid, calcium stearate, sodium bicarbonate, or mixtures thereof. More preferably the lubricating agent is magnesium stearate or stearic acid.

Preferably the disintegrating agent includes microcrystalline cellulose, maize starch, sodium starch glycollate, low substituted hydroxypropyl cellulose, alginic acid or croscarmellose sodium, sodium bicarbonate, or mixtures thereof.

Preferably the binder includes polyvinyl pyrrolidone, gelatin, Gelucire®, hydroxypropylmethyl cellulose, starch, or mixtures thereof.

Suitable flow aids include, but are not limited to talc and colloidal silicon dioxide.

Liquid fill compositions (for example, viscous liquid fills, liquid paste fills, or thixotropic liquid fills) are also suitable for oral administration. Melt filled compositions may be obtained by mixing meloxicam with certain esters of natural vegetable oil fatty acids, for example, the Gelucire® range available from Gattefosse to provide a variety of release rates. Suitably a melt-filled capsule comprises a) 10-80% meloxicam and b) 20-90% of a fatty acid ester excipient which comprises one or more polyol esters and triglycerides of natural vegetable oil fatty acids.

Suitable pharmaceutically acceptable hydrophobic carriers include the glycerides and partial glycerides. The preferred carriers are known under the trademark Gelucire®, and are commercially available from Gattefosse Corporation; Hawthorne, N.Y. Gelucires® are available with varying physical characteristics such as melting point, HLB and solubilities in various solvents. The preferred Gelucire® is Gelucire® 44/14.

For example, a tablet of the present invention may include 1-99% of meloxicam; about 10 to about 60% by weight of a bicarbonate; and 20-90% of a fatty acid ester excipient which comprises one or more polyol esters and triglycerides of natural vegetable oil fatty acids. The use of esters of fatty acids, e.g., Gelucire®, is well known to those skilled in the art, as is evident from the number of patents that disclose its use. Exemplary patents include, but are not limited to U.S. Pat. No. 6,361,796; U.S. Pat. No. 6,312,704; U.S. Pat. No. 6,251,426; U.S. Pat. No. 6,242,000, and U.S. Pat. No. 6,238,689, among many others.

The compositions of the present invention may additionally comprise a taste masking component for example a sweetener, a flavoring agent, arginine, sodium carbonate or sodium bicarbonate.

Solid non-effervescent compositions are preferred compositions of the present invention. In the most preferred compositions and methods, a small degree of effervescence may occur in the stomach, leading to disintegration of the tablet. In the most preferred compositions and methods, effervescence does not include or involve dissolution of the active ingredient. The preferred compositions are preferably formed into a tablet.

In the compositions of the present invention the NSAID, such as meloxicam, may, if desired, be associated with other compatible pharmacologically active ingredients and/or enhancing agents. Thus, for example, meloxicam may be combined with any ingredient commonly used in a cough or cold remedy, for example, an antihistamine, caffeine or another xanthine derivative, a cough suppressant, a decongestant, an expectorant, a muscle relaxant, or combinations thereof. Exemplary compatible pharmacologically active ingredients include, but are not limited to codeine, oxycodone, hydrocodone, and/or hydromorphone.

Suitable antihistamines which are preferably non-sedating include acrivastine, astemizole, azatadine, azelastine, bromodiphenhyrdramine, brompheniramine, carbinoxamine, cetirizine, chlorpheniramine, cyproheptadine, dexbrompheniramine, dexchlorpheniramine, diphenhydramine, ebastine, ketotifen, lodoxamide, loratidine, levocubastine, mequitazine, oxatomide, phenindamine, phenyltoloxamine, pyrilamine, setastine, tazifylline, temelastine, terfenadine, tripelennamine or triprolidine. Suitable cough suppressants include caramiphen, codeine or dextromethorphan. Suitable decongestants include pseudoephedrine, phenylpropanolamine and phenylephrine. Suitable expectorants include guaifensin, potassium citrate, potassium guaiacolsulphonate, potassium sulphate and terpin hydrate.

In another aspect the present invention provides a method of preparing a pharmaceutical composition comprising meloxicam together with sodium bicarbonate as an absorption aide. Meloxicam and bicarbonate are administered in a solid dosage form which upon exposure to stomach juice they start to react to one another. This provides first disintegration, second, motion and third, increased solubility. The increased solubility is maintained by the presence of gelucire.

In a further aspect the present invention provides a process to prepare a pharmaceutical composition comprising meloxicam and a disintegrating agent, together with a pharmaceutically acceptable carrier comprising combining meloxicam in solid form with a pharmaceutically acceptable carrier and formulating into a dosage form.

A preferred process for preparing a solid composition in tablet form comprises combining 10-90% of meloxicam with 1-90% of a diluent, optionally adding other pharmaceutically acceptable excipients selected from lubricating agents, disintegrating agents, binders, flow aids, oils, fats and waxes, mixing the ingredients with one another to form a uniform mixture, and compressing the mixture thus obtained to form tablets which may be optionally coated with a film coat or a sugar-coat.

In a most preferred embodiment of the invention, the present invention provides a process for preparing an Meloxicam-containing formulation comprising the steps of: dissolving meloxicam in an alkaline solution; mix with Neusilin®; change the pH (e.g., by adding glacier acid) so that the meloxicam re-crystallizes; and remove the solution. The meloxicam is thereby loaded on the Neusilin® matrix. It should then be dried, e.g., overnight. The meloxicam loaded on the Neusilin® can then be mixed with one or more ingredients according to the invention. For example, it can be mixed with tartaric acid, corn starch, Gelucire®, sodium bicarbonate, microcrystalline cellulose, and sodium croscarmellose. The composition is then suitable for compressing into a tablet.

In the absence of moisture, fine particles of the NSAID, preferably Meloxicam, bicarbonate, preferably sodium bicarbonate, Gelucire®, preferably grade 44/14, and optionally other excipients are thoroughly mixed and converted into granules. Granules may be packaged as individual doses or may be compressed under low compression pressure to form tablets.

The mixing of the ingredients may be achieved in different ways. One way is to mix the NSAID and bicarbonate and placed them in a fluidized bed system and while mixing, spray a solution of Gelucire® dissolved in a suitable vehicle preferably isopropanol onto the suspending dry mixture. Another method is to melt a mixture of Gelucire® and the NSAID at the lowest possible temperature and after drying of the mixture mix well with bicarbonate in the presence or absence of a suitable solvent preferably isopropranol.

The granulates obtained according to the above described methods are then screened, dried, combined with bicarbonate and any selected excipient(s) in the desired amounts and compressed in suitable molds for obtaining the desired tablets which can then be film coated, if desired.

In addition to good handling and workability, the tablets of the present invention provide complete dissolution of the active ingredient in about 10 minutes or less, preferably in less than about 5 minutes. Consequently the release is faster with respect to the commercially available meloxicam based analgesic tablets.

One skilled in the art readily recognizes that tablet compression provides certain benefits and characteristics in the administration and presentation of an active ingredient for adsorption. It is also known to those skilled in the art that the exact composition of a tablet partially dictates the method and attributes of the compression process. For example, it is generally known that too much compression may slow the release or disintegration of the tablet into smaller particles. It is therefore an embodiment of the invention to provide a tablet having been compressed within a range of compression values that promote or do not adversely affect disintegration of the tablet at the enhanced rate that forms an embodiment of the present invention.

It will be appreciated by the person skilled in the art that due to the different excipients used in the formulation and varying amounts thereof that for any compression pressure, different formulations will have different crushing strengths and disintegration times. Preferred dosage forms exhibit a crushing strength of 6.5-15 Kp and a disintegration time of less than 10 minutes at a compression force above 80 MPa. More preferred formulations exhibit a crushing strength of 6.5-15 Kp and a disintegration time of less than 10 minutes when compressed at a compression force in the range 100-140 MPa such as by a standard tabletting machine, e.g. a rotary tabletting machine. Such compression pressures include, 110 MPa, 120 MPa and 130 MPa. Especially preferred dosage forms exhibit a crushing strength of 6.5-15 Kp and a disintegration time of less than 10 minutes when compressed at all pressures in the range 100-140 MPa.

The disintegration time of the tablet formed in accordance with the present invention is less than 10 minutes as measured by the method described in the European Pharmacopoeia 1986, Ref V.5.1.1 (updated 1995) (A. Disintegration Test for Tablets and Capsules). Preferred disintegration times are less than 6 minutes (e.g. 1-6 minutes), more preferably less than 5 minutes (e.g. 1-5 minutes) and most preferably 3 minutes or less (e.g. 1-3 minutes).

As used herein, a diluent or filler is used in its conventional pharmacological definition, and refers to an ingredient that adds necessary bulk to a formulation to prepare tablets of a desired size.

As used herein, a binder or adhesive is used in its conventional pharmacological definition, and refers to an ingredient that promotes the adhesion of the particles of the formulation.

As used herein, a disintegrator or disintegrating agent is used in its conventional pharmacological definition, and refers to an ingredient that promotes the post-administration break-up of the tablets into smaller particles for more ready drug availability.

As used herein, a lubricant or lubricating agent is used in its conventional pharmacological definition, and refers to an ingredient that enhances the flow of the tabletting material into the tablet dies, and prevents the tabletting material from sticking to punches and dies.

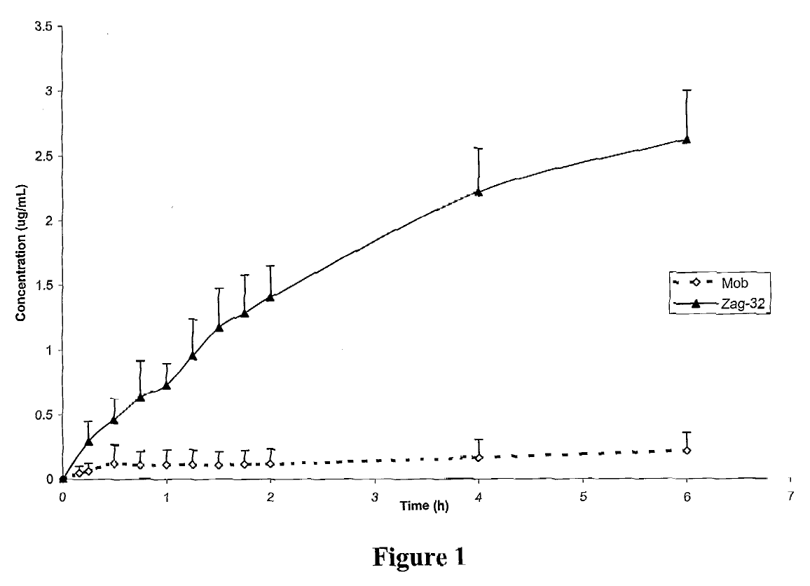

As used herein, enhanced absorption or similar terms and phrases relating to the relative speed, rate, and/or quantity of the bioavailability of the active agent. In accordance with the present invention, enhanced absorption is measured in reference to the standard in the industry, Mobicox®. In essence, the compositions of the present invention provide, to a patient in pain, a greater concentration of active agent faster, as compared to the bioavailability curve for Mobicox®. In graphical or mathematical terms, enhanced absorption may be determined or quantified by using the area under the curve (AUC). As shown in FIG. 1, the extent and rate of absorption, as represented by the AUC, for the formulations of the present invention, delivers a greater amount of active agent in a shorter time frame as compared to Mobicox®. In accordance with the teachings of the present invention, it is important to determine enhanced absorption of a particular composition as it applies to a patient in pain, or data obtained from a patient or subject in pain.

In therapeutic use the dosage forms of the present invention are administered orally, thus the therapeutic dosage forms are presented in solid dosage form, preferably as a tablet. The dosage forms may be uncoated or coated with a sugar or film coating, which dissolves substantially immediately the dosage form comes into contact with an aqueous medium. The composition may also be compressed onto a solid core of another material to form a solid formulation with an quick release outer coating. Alternatively, the compressed composition may be present in one or more layers of a multi-layer solid dosage form. In such formulations the remaining layers or core may comprise standard excipients to provide conventional, fast or slow release and are well within the knowledge of a person skilled in the art (e.g., see Remington's Pharmaceutical Sciences, 17th Edition, Ed Gennaro et al; or Ansel=s Aintroduction to Pharmaceutical Dosage Forms@, 2^{nd} edition, Henry Kimpton Publishers).

The following Examples illustrate specific formulations comprehended by the present invention, and methods for their preparation. The Examples are not intended to be limiting to the scope of the invention in any respect and should not be so construed.

### EXAMPLES

### Example 1

### Animal Model

Delayed absorption caused by vagal suppression that has previously been reported in the literature (e.g., Jamali & Axelson, 1997) was used to test the absorption rates of new meloxicam formulations.

The animal models are adult male Sprague-Dawley rats with body weight of 250-300 g, and which were cared for in accordance with the principles and guidelines of the Canadian Council of Animal Care. All rats were catheterized in the right jugular vein for sample collection.

An animal model having suppressed vagal properties were produced by administering (intraperitoneal injection) to the rats two 20 mg/kg doses of propantheline (test, n=6), an anticholinergic agent with known vagal suppressive properties, the first dose at 2 hours prior to administration of an NSAID, and the second at 1 hour prior.

One hour after the second dose of propantheline, 20 mg/kg doses of a commercially available meloxicam tablet (Motrin 200 mg tablets, available from McNeil, Guelph, Canada, KIN 02186934, Batch 151979/(L)F316/Exp March 2001) were administered. The tablets were crushed gently and small pieces were administered into the stomach via a plastic tube followed by 0.5 mL tap water. Animals were fasted after the first dose of propantheline until 4 hours post-meloxicam dose. They had free access to water.

Serial blood samples were withdrawn from the jugular vein cannula at suitable times post-ibuprofen dose. Plasma was separated and kept at −20_C until analyzed for ibuprofen using a high performance chromatography method (Wright et al, 1992).

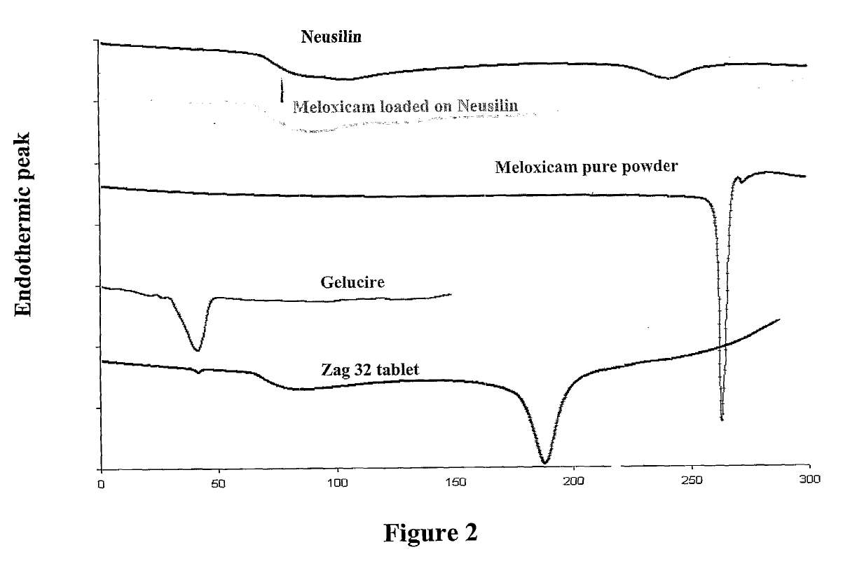

Results. Table 1 and FIG. 2 show that the absorption rate for ibuprofen in a vagally suppressed rat model was suppressed similar to what is reported in humans (Jamali & Kunz, 1999). Propantheline treatment (i.e., vagal suppression) caused a substantial and significant delay in absorption of ibuprofen. Notably, AUC(0-1), a reliable measure of absorption-rate was significantly reduced from 48.7 to 12.2 μg/h/mL.

TABLE 1
Bioavailability indices following oral administration
of 20 mg/kg of ibuprofen as crushed tablets to control
and vagal-suppressed (Pain Model) rats.

| | Tmax | Cmax | AUC (0-1) | AUC (0-8) |
|---|---|---|---|---|
| Rats | hour | μg/mL | μg/h/mL⁻¹ | μg/h/mL⁻¹ |
| Control | 0.28 | 40.4 | 48.7 | 139 |
| Pain Model | 0.75 | 13.8* | 12.2* | 81.8 |

*significantly different from Control (a = 0.05)

### Example 2

The oral bioavailability of meloxicam (MEL) from a commercially available tablet (noted below as “Brand”) was incomplete in healthy rats. Bioavailability is expected to be even lower under simulated acute pain condition when the vagal nervous system is suppressed. This example shows the development of a MEL formulation with improved oral absorption using a vagally suppressed rat model mimicking acute pain conditions.

Methods: Tablets (“TEST”) were made by loading MEL on magnesium aluminum silicate (Neusilin®) and mixing with Gelucire® 44/14. The solubility and dissolution rate of TEST and BRAND were evaluated in simulated gastric fluid (pH 1.2). The plasma concentration of MEL was assessed following IV solution (in 5 mM NaOH) and BRAND in control rats, as well, after TEST and BRAND in vagally suppressed (20 mg/kg ip propantheline 2 and 1 h before dosing) rats.

Both TEST and BRAND tablets were gently crushed and administered (0.83-1.03 mg/kg MEL) via a plastic gastric lavage tube followed by 0.5 mL water. Serial blood samples were collected via a catheter inserted in the right jugular vein. MEL was assayed using HPLC.

Results: In 60 min, 8.7_{—}0.2% and 40.7_{—}2.1% of MEL were released from BRAND and TEST, respectively. Solubility at pH 1.2 for MEL contained in BRAND and TEST was 1.2 and 359 mg %, respectively. AUC data during the first 1 and 6 h post-dose (adjusted based on 0.9 mg/kg) were significantly reduced by vagal suppression. AUC of TEST was significantly greater than BRAND.

AUC, μg · h mL⁻¹

| Route | Formulation, Status | 0-1 h | 0-6 h |
|---|---|---|---|
| I.V. | MEL, Control | 5.96 ∀ 0.97 | 25.85 ∀ 4.34 |
| Oral | BRAND, Control | 0.32 ∀ 0.21 | 10.02 ∀ 5.9 |
| | BRAND, Treated | 0.10 ∀ 0.10^{a} | 0.95 ∀ 0.81^{a} |
| | TEST, Treated | 0.42 ∀ 0.16^{b} | 10.0 ∀ 0.85^{b} |

^{a}Different from BRAND Control;
^{b}Different from BRAND Treated (p < 0.05)

Conclusion: Incorporating Neusilin® and Gelucire®44/14 in solid dosage formulations of meloxicam significantly improves solubility and dissolution rate. In a vagally suppressed animal model that mimics a human in pain, this formulation resulted in an increased rate and extent of absorption of meloxicam in the acidic environment.

### Example 3

TABLE 2
Composition of three different types
of meloxicam-loaded-Neusilin ®

| Different types of | | |
|---|---|---|
| meloxicam-loaded-Neusilin ® | % of Meloxicam | % of Neusilin ® |
| ZM-Neu1 | 16.7 | 83.3 |
| ZM-Neu2 | 33.3 | 66.7 |
| ZM-Neu3 | 46.8 | 53.2 |

TABLE 3
Recipe for a meloxicam formulation tablet

| | Amount | % of total | % of |
|---|---|---|---|
| Name of Ingredient | (mg) | mass of tablet | meloxicam |
| ZM-Neu3 | 214* | 28.01 | 13.09 |
| Na Bicarbonate | 168 | 21.99 | 0 |
| Gelucire ® 44/14 | 38.2 | 5.00 | 0 |
| Tartaric Acid | 76.4 | 10.00 | 0 |
| Microcrystalline cellulose | 114.6 | 15.00 | 0 |
| Corn Starch | 114.6 | 15.00 | 0 |
| Na Cross Carmalose | 38.2 | 5.00 | 0 |
| Total** | 764 | 100 | 13.09 |

*This contains 100 mg of Meloxicam
**A tablet with a total weight of 115 mg will contain 15 mg of meloxicam

### Example 4

Various ingredients of the meloxicam formulation described in Table 3 were tested to determine any variation in meloxicam's endothermic peak. FIG. 2 illustrates graphically the results of testing Neusilin® alone, meloxicam as a pure powder, Gelucire® alone, and a meloxicam formulation of the present invention (“Zag 32”). The tests showed that meloxicam's endothermic peak was altered when formed into a tablet having the formulation shown in Table 3.

### Example 5

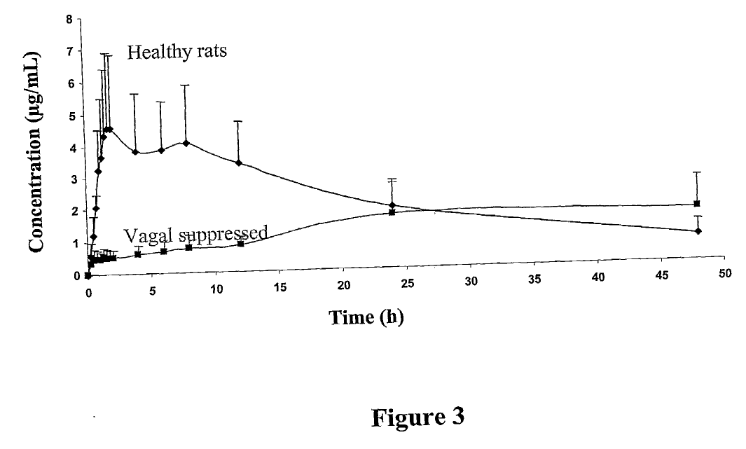

A formulation of meloxicam commercially available under the trademark Mobicox®, was tested for absorption in a rat model that is characterized by the traits of an animal or human in pain. The rat model is one that has a suppressed Vagus nervous system, and the rate of absorption was determined as plasma concentration over time. FIG. 3 shows the plasma concentration-time profiles after oral administration of meloxicam (0.9 mg/kg) as Mobicox® to healthy and pain-mimicking rats (n=5). As shown in FIG. 3, the rate of absorption of Mobicox® was significantly decreased in vagally suppressed rats as compared to healthy rats.

### Example 6

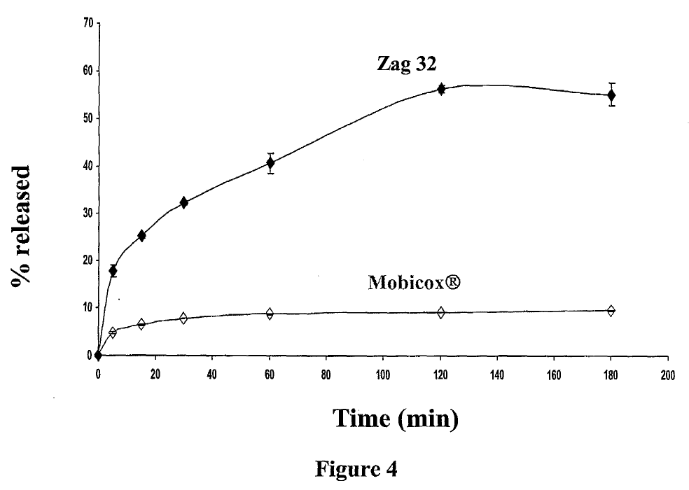

FIG. 4 shows that dissolution of meloxicam was substantially improved in a formulation of the present invention as compared to a commercially available formulation. The formulation of the present invention (“Zag 32”) is the formulation shown in Table 3, and represents a meloxicam formulation in which meloxicam is loaded on Neusilin® and then mixed with Gelucire® 44/14.

### Example 7

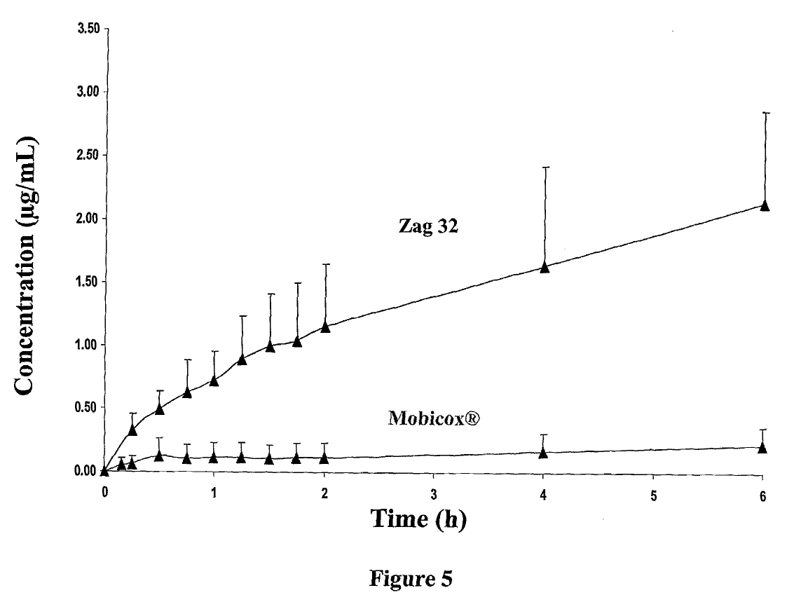

The oral availability of a meloxicam formulation of the present invention (Zag 32; also shown in Table 3) was compared to a commercially available formulation (Mobicox®). The plasma concentration-time profile of meloxicam was tested after orally administering to rats meloxicam (0.9 mg/kg) in a Zag 32 formulation (n=7) as compared to Mobicox® (n=5). The Zag 32 formulation included meloxicam loaded on Neusilin® and mixed with Gelucire® 44/14. FIG. 5 shows that the oral availability for Zag 32 was substantially improved as compared to Mobicox®.

### Example 8

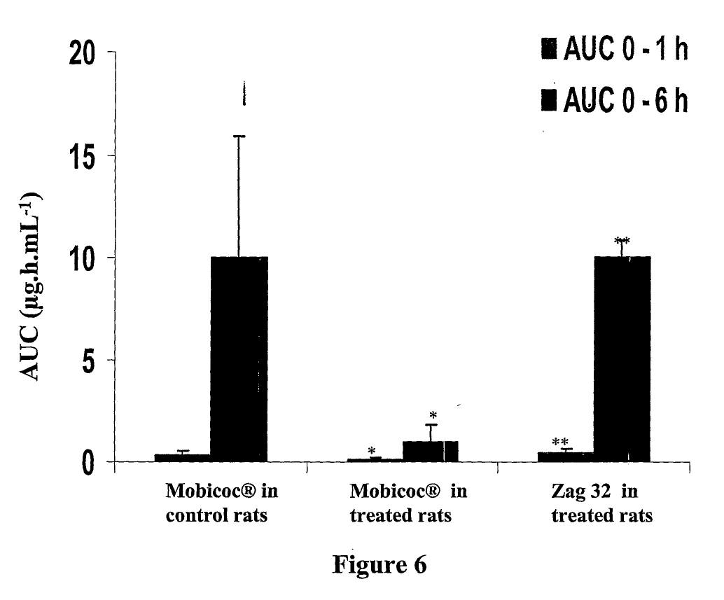

Two meloxicam formulations were compared to determine the amount and rate of absorption in terms of area under the curve (AUC). Control and treated rats were administered tablets containing 0.9 mg/kg meloxicam for a composition of the present invention (Zag 32″) and a commercially available formulation (Mobicox®). The results are shown in FIG. 6. As illustrated, * refers to those different from Mobicox® control group (p>0.05), and ** refers to those different from Mobicox® treated group (p>0.05).

### Example 9

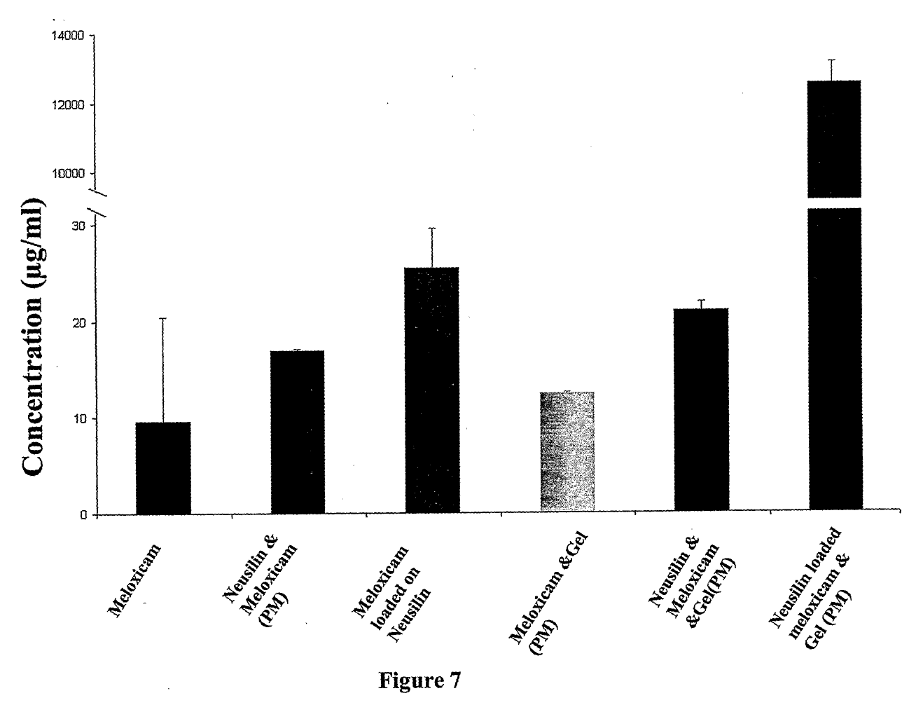

The solubility of meloxicam in simulated gastric FLUID (pH 1.2) was compared for various formulations of meloxicam. FIG. 7 shows that the solubility was substantially increased when it was loaded on Neusilin® and mixed with Gelucire® 44/14.

### Example 10

At pH 1.2, solubility of meloxicam is 7 mg/L. Gelucire® and the metal silicate alone increased solubility of meloxicam to 10 mg/L and 23-41 mg/L, respectively. The combination of Gelucire® and Neusilin® unexpectedly increased solubility to 11530 mg/L. The combination resulted in a 10-fold increase in oral bioavailability within 6 hours post-administration that translates to many fold further overall (0-infinity) increase in bioavailability in the rat. The unexpected increase in solubility does not extend to ibuprofen.

### Example 11

### Discussion and Conclusions

Comparison of the DSC thermograms of meloxicam alone with that loaded on Neusilin® suggests an interaction between the drug and the excipient.

Neusilin®/Gelucire® formulation of meloxicam, has increased water solubility, dissolution and oral bioavailability as compared with the commercially available formulation.

The improved-properties of meloxicam formulation may be attributed to the solubilizing properties of Gelucire®, alkaline nature of Neusilin®, and amorphous state of the loaded meloxicam.

This is suggested to be due to suppression of the vagus nervous system resulting in reduced gastrointestinal motility and fluid secretion, hence, reduced disintegration and dissolution.

Objectives: to develop rapidly absorbed formulations of meloxicam using a rat model of vagal suppression.

### Materials and Methods:

Preparation meloxicam tablet (ZAG 32, patent pending) were prepared, by loading of meloxicam on magnesium aluminum silicate (Neusilin® US2) and mixing with Gelucire® 44/14.

Differential scanning calorimetry—DSC analyses were performed using a SSC/5200 SII DSC analyzer. The experiments were done in a sealed aluminum pan; the weight of each sample was 5±mg and the heating rate was 10° C./min.

### Solubility Measurements

Excess of different compositions and pure meloxicam powder were transferred into test tubes.

10 ml of USP simulated gastric fluid, pH 1.2 (SGF) was added.

Samples were shaken for 72 h at room temperature.

Filtered solutions were analyzed for meloxicam.

### Animal Study

Male, Sprague-Dawley rats (n+17) were cannulated in the right jugular vein, and allowed to recover overnight.

Vagal suppression was achieved by intraperitoneal injection of two propantheline doses.

The Zag 32 and Mobicox® (Boehringer Ingelheim) tablets were gently crushed and the dry granules orally administered (0.9 mg/kg of meloxicam) with 0.5 mL water to control and suppressed rats. They were kept fasted until 4 h post-dosing with free access to water.

### Dissolution Test

USPXXII paddle method at 37°, 75 rpm in simulated gastric fluid (pH 1.2)

Amount released was measured with a UV spectrophotometer at 364 nm.

Drug release profile from Zag 32 formulation was compared with Mobiocox (Boehringer Ingelheim) tablets 15 mg.

HPLC: Drug plasma concentrations were determined using a previously reported HPLC method.

Although the present invention has been described in terms of particular preferred embodiments, it is not limited to those embodiments. Alternative embodiments, examples, and modifications which would still be encompassed by the invention may be made by those skilled in the art, particularly in light of the foregoing teachings.

1. A pharmaceutical composition comprising a non-steroidal anti-inflammatory active agent, and an alkalating agent.

2. The pharmaceutical composition of claim 1 wherein the active agent is selected from the group consisting of piroxicam, meloxicam, indomethacin, fenoprofen, keterolac, naproxen, and ibuprofen.

3. The pharmaceutical composition of claim 1 further comprising at least one disintegration agent, at least one anti-precipitation agent; and at least one excipient.

4. The pharmaceutical composition of claim 3 wherein the disintegration agent is an alkali metal carbonate.

5. The pharmaceutical composition of claim 4 wherein the alkali metal carbonate is sodium bicarbonate.

6. The pharmaceutical composition of claim 3 wherein the anti-precipitation agent is a fatty acid ester.

7. The pharmaceutical agent of claim 6 wherein the fatty acid ester is gelucire.

8. The pharmaceutical composition of claim 3 wherein the excipient is tartaric acid.

9. The pharmaceutical composition of claim 1 comprising meloxicam, Neusilin®, Gelucire®, sodium bicarbonate, tartaric acid, and starch.

10. The pharmaceutical composition of claim 9 further comprising hypromellose, pre-gelatinized starch, microcrystalline cellulose, sodium croscarmellose, and magnesium stearate.

11. A method for the treatment of inflammation comprising supplying an anti-inflammation formulation, said formulation comprising a non-steroidal anti-inflammatory active agent, and an alkalating agent.

12. The method of claim 11 wherein the active agent is meloxicam and the alkalating agent is Neusilin®.

13. The method of claim 12 further comprising a disintegrating agent comprising an alkali metal carbonate, an anti-precipitation agent, and an excipient comprising tartaric acid; and administering said formulation.

14. A pharmaceutical composition comprising meloxicam, Gelucire®, and Neusilin®.

15. The pharmaceutical composition of claim 14 further comprising sodium bicarbonate, tartaric acid, and corn starch.

16. A method for enhancing the solubility of meloxicam comprising loading meloxicam on a silicate matrix, and mixing the matrix containing meloxicam with a fatty acid ester.

17. The method of claim 16 wherein the silicate matrix is Neusilin®.

18. The method of claim 16 wherein the fatty acid ester is Gelucire.
